(19) United States
(12) Patent Application Publication
(10) Pub. No.: US 2026/0120853 A1
(43) Pub. Date: Apr. 30, 2026

(54) Healthcare Charge Capture and Prioritization System and Method

(71) Applicant: DPACC Administrative Services, LLC, Dallas, TX (US)

(72) Inventors: Tung Tran, Southlake, TX (US); Shibu Thomas, Plano, TX (US); Amit Mann, Lewisville, TX (US); Melissa Stewart, Hurst, TX (US); Leia Oh-Spradling, Richardson, TX (US); Stephanie Fargo, Hurst, TX (US)

(21) Appl. No.: 19/004,642

(22) Filed: Dec. 30, 2024

Related U.S. Application Data
(63) Continuation-in-part of application No. 18/059,940, filed on Nov. 29, 2022, now Pat. No. 12,217,858.
Application No. 18/059,940 is a division of application No. 18/059,933, filed on Nov. 29, 2022, now Pat. No. 12,183,455.
Application No. 18/059,933 is a continuation of application No. 16/743,625, filed on Jan. 15, 2020, now Pat. No. 11,972,860.

(51) Int. Cl. G16H 40/20; G16H 15/00

(52) U.S. Cl. CPC G16H 40/20; G16H 15/00

(57) ABSTRACT

A healthcare charge capture and prioritization system for assisting healthcare providers with properly and accurately capturing procedures and prioritizing code selection for claim submission, generally. The present invention that is a charge capture system can analyze healthcare claim-related data and usage data for ranking and ordering Current Procedural Technology (CPT) and International Statistical Classification of Diseases and Related Health Problems (ICD) codes to reduce the utilization of additional processing resources and network utilization from repeated system denials. Additionally, the present disclosure can generate and track analytics relevant to patient encounters and provider action for processing and managing patient interactions, processing and managing healthcare-related charge entry, processing and prioritizing healthcare-related procedure technology codes, and processing and prioritizing disease and related health problem codes for prioritized and efficient selection by a provider.

### REFERENCE TO RELATED APPLICATIONS

The present application is a Continuation-in-Part of U.S. Non-Provisional application Ser. No. 18/059,940, which is a Co-Divisional Application of U.S. Non-Provisional application Ser. No. 18/059,933, both filed Nov. 29, 2022, both being a continuation of U.S. Non-Provisional application Ser. No. 16/743,625 filed on Jan. 15, 2020, each of which is incorporated by reference in their entirety.

### FIELD OF THE INVENTION

The present invention generally relates to healthcare charge capture systems and method of use, more particularly to charge capture systems having prioritization algorithms, code preferencing, time tracking functions, activity logging, enhanced billing functions, or a combination thereof, for the creation of efficiencies in charge capture and claims submission.

### BACKGROUND OF THE INVENTION

Generally speaking, charge capture systems provide a means for service providers to track and manage interactions with patients. However, with multiple providers (both physicians and Advanced Practice Providers or “APPs”) engaging multiple patients at multiple facilities over the course of a day, services and time may go unaccounted, procedures may go unidentified or misidentified, charge entry can be put on the backburner, claims may be rejected as improper or untimely, and efficiencies may compound and exacerbate one another—potentially resulting in wasted resources and lost revenue if the charges are not billed timely or at all.

Additionally, charges for procedures can be as subjective as the review of the charges for payment. Two mechanisms have been standardized in an attempt to remove the subjectivity of procedures and disease states: International Statistical Classification of Diseases and Related Health Problems (ICD) codes and Current Procedural Technology (CPT) codes. In short, the ICD codes and CPT codes tell the payer that a particular patient at a particular healthcare facility exhibits certain symptoms (represented by the ICD codes) and that the providers performed certain procedures to treat those symptoms (represented by the CPT codes carrying a monetary value in RVU units). These codes tell the insurance payer what diagnosis is attributable to a patient's symptoms, what procedures the healthcare provider completed and, finally, what services the provider would like to be reimbursed for.

However, when claims, having ICD codes, are submitted to the payer for approval or denial, codes, although related, may be deemed not required or authorized for a particular disease state which may then be denied. If a claim leads with these (ancillary) procedure codes, the claim could be denied before the essential procedure codes are reviewed. Similarly problematic is the submission of a CPT code for a lower paying procedure, when a higher paying procedure is available—such as procedures that are performed by physicians, which generally pay at higher rates than procedures performed by APPs. This is both inefficient and creates unnecessary costs.

Moreover, certain features of an efficient charge capture system, in addition to ICD codes and CPT codes, may be leveraged to further refine the current system as to ensure (1) charge capture data is secured accurately and (2) inefficiencies are minimized (or the in the alternative, efficiencies are maximized) as to generate claims which more precisely reflect the care received by the patient and supplied by the provider.

Too, attendant to the above CPT and ICD prioritization procedures, a preferencing of control logic-selectable, provider-selectable or administratively-selectable ICD codes and/or CPT codes may be utilized to selectively increment codes preferentially selected for and selectable by a particular provider (e.g., cardiology-related codes for a cardiologist, respiratory-related codes for a pulmonologist), codes having a high rate of recurrence or use by a single provider, those codes which are specific for a certain location or facility (e.g., urgent care, critical care or clinic setting), or those codes, specific or non-specific to a provider type or particular physician, where ICD and/or CPT codes have been incremented as to ensure the accurate measure of time (as above), higher claim acceptance, fewer claim rejections, or a combination thereof.

One other of these efficiency-facilitating factors is the tracking of total number of minutes and hours spent relative to each encounter with a patient on a provider-by-provider basis (i.e., time). This time component may be sequential or contemporaneous and may also involve one to a plurality of providers (e.g., physicians, nurse practitioners (NPs), Advanced Practice Nurses (APN)s, nurses and/or other ancillary medical professionals). As well, each unit or plurality of units of time are not fungible across all providers wherein considerable disparity may exist across and among providers as a weighted measure (time x rate/quantity x quality) which must be calculated precisely typically as a ratio of several provider inputs as to construct accurate billing, generate fewer chargebacks and ensure medical resources are properly provided to the patient.

### SUMMARY OF THE INVENTION

The present disclosure achieves technical advantages as a healthcare charge capture and prioritization system for assisting healthcare providers with capturing their procedures and prioritizing code selection for claim submission. The present disclosure solves the technological problem of applying a data optimization algorithm, together with the incorporation of time measuring and selective and preferential code display functions to healthcare charge-related data to generate and present optimized electronic claims via an improved healthcare charge analytic system.

The present disclosure improves the performance of the healthcare charge analytic system itself by analyzing healthcare claim-related data for ranking and ordering Current Procedural Technology (CPT) and International Statistical Classification of Diseases and Related Health Problems (ICD) codes, together with a time ordering, management and accounting functions, which is then preferentially presented to providers as a subset of possible or potential code selections for ranked and ordered CPT and ICD codes, based on claim-related data, to reduce the total selectable CPT and ICD codes to those codes which are “preferred”, “preferenced” or “favorite” as to reduce overall strain and unnecessary utilization of additional processing resources and network utilization from denied, rejected or decremented CPT and ICD codes based on repeated system claim denials and/or rejections. Additionally, the present disclosure can generate, and track analytics and increment selectable code choices most relevant, most utilized, having a higher probability of acceptance or most pertinent to patient encounters and provider actions.

In contrast, traditional charge capture systems simply reroute existing data, without analysis, prioritization or selective presentation, resulting in haphazard and ineffectual claim submissions that do not advance the claims process, but instead simply add to the strain on an already overspent system. Advantageously, the present disclosure provides advancements which enhances the healthcare claim process by implementing algorithms that capture and weigh various claim data to create a database of charge data analytics and present those codes which have an increased probability of third-party acceptance, those claims which are more closely tailored to represent the care a patient receives, and/or those codes which are provider-specific, practice-specific or location-specific, or otherwise preferenced that can streamline charge capture and claim generation functionalities for the healthcare provider by reducing unnecessarily broad and expansive code choices to a preferenced subset of code choices. Importantly, the present disclosure can save a provider time, more accurately account for time, increase revenue, provide a platform for previously uncaptured data analytics, increase patient throughput, provide a higher level of patient care, improve patient satisfaction, and identify and address areas for improvement.

It is an object of the present invention to provide a system for processing and managing patient-provider interactions. It is a further object of the invention to provide a system for processing and managing healthcare-related charge entry. It is a further object of the invention to provide a system of preferred selectable ICD and CPT codes for processing and prioritizing healthcare-related procedure technology codes after ICD and CPT selection and provider type based on claim acceptance, claim rejection and reimbursement rates and for providing a truncated ICD and CPT selectable list for claim submission before submission has been attempted based on prior claim acceptance or rejection, provider preference, provider utilization, provider type, facility type or other like criteria. It is a further object of the invention to provide a system for processing and prioritizing disease and related health problem codes based on previous order and ranking through a feedback loop. It is yet another object of the invention to account for provider time as a function of the current healthcare-related charge entry system. It is still another object of the present invention to provide prioritized, preferenced or incremented codes for provider selection. It is still another object of the present invention to provide for additional efficiencies through a user interface tracking activity logs and to provide more efficient billing functions These and other objects are provided by the following embodiments as defined and detailed by the present disclosure.

In one embodiment, a system for processing and managing patient interactions, can include: a system processor running a patient interaction module configured to, in order to process and manage patient interactions and notifications: receive login credentials from a user device for a user over an encrypted network; receive patient data for a plurality of patients at a first location from an electronic health record system for patients assigned to the user for a predetermined time period over an encrypted network; determine a number of patient encounters for the user for the predetermined time period and generate an indication of the number of patients seen and unseen, and a progress bar representing a completion percentage of the number of patients seen for display on a user device; generate a notification indicating those patients having outstanding encounters for display on the user device; generate a patient record for each patient using at least a portion of the received patient data, including an encounter indicator, indicating whether the patient has been seen by a physician, seen by an advanced practice provider, or needs to be seen, and a clinical indicator; generate a patient profile having the encounter indicator, at least a portion of the received patient data, a summary of the patient's condition, and a follow-up task listing for display on the user device; and provide a user interface for modifying the patient profile to the user device over an encrypted network.

In another embodiment, a Current Procedural Technology (CPT) prioritization platform, can include: a server having a system processor running a healthcare charge entry module configured to, in order to process and prioritize a CPT code: receive CPT information for a patient for a predetermined period, including provider information and patient information; determine whether the provider for each received CPT is a physician or APP; determine whether multiple CPTs occurred over the predetermined period; associate, via the server, a work relative value unit (RVU) for each received CPT; compare the CPT RVUs, via the server, to rank the CPT RVUs over the predetermined period; if there is a tie for the highest CPT RVU value, select a physician CPT with the highest RVU for claim submission, otherwise, select a CPT with the highest RVU for claim submission; and generate a claim for the predetermined period using the selected CPT.

In another embodiment, an International Statistical Classification of Diseases and Related Health Problems (ICD) code prioritization system, can include: a server having a system processor running a healthcare charge module configured to, in order to process and prioritize disease and related health problem codes: receive an approval or rejection of one or more ICD codes in the submitted claim; if an ICD code is approved, increment an ICD rank for the approved ICD code and increment an approval count, if an ICD code is denied, decrement the ICD rank for the denied ICD code and increment a denial count; store the ICD rank, the incremented count, and a timestamp of the approval or disapproval for the received approval or rejection; retrieve a history of the approved ICD code for a predetermined time frame; if a number of approved ICD codes surpasses a first threshold, increment the ICD rank for the approved ICD code, otherwise, analyze the ICD code approval amount; if the ICD code approval amount surpasses a second threshold, increment and store the ICD rank for the approved ICD code, otherwise, store the ICD code rank.

In another embodiment, a system for processing and managing patient interactions can include: the selection and display of preferred or “favorite” CPT and ICD codes, and/or care locations, for presentation to a provider wherein individual codes, or sets or subsets of codes, may be preferably displayed for selection from all or a subset of available codes (CPT codes, ICD codes, CLSLs, or a combination thereof) from a larger set of codes which may be selected from this larger code or location set based on provider use, provider type, care location, location type (which may be virtual), incremented codes, reimbursement rates, or a combination thereof, based on one or a number of factors including (a) utilization, (b) specialty field, (c) a specific provider, (d) a provider type, (e) congruence with a billing module, (f) billing specialists or provider (self) population, (g) auto-population algorithmically through control logic based on accepted or rejected claims, (h) provider location, (i) and/or quick and easy access.

In yet another embodiment, a system for processing and managing patient interactions, can include: a system processor running a patient interaction module configured to, in order to process and manage patient interactions and notifications: receive login credentials from a user device for a user over an encrypted network; receive patient data for a plurality of patients at a first location from an electronic health record system for patients assigned to the user for a predetermined time period over an encrypted network; determine a number of patient encounters for the user for the predetermined time period and generate an indication of the number of patients seen and unseen, and a progress bar representing a completion percentage of the number of patients seen for display on a user device; track and calculate time, in minutes, hours, or a combination thereof, units of time supplied by a provider or providers; generate a notification indicating those patients having outstanding encounters for display on the user device; generate a patient record for each patient using at least a portion of the received patient data, including an encounter indicator, indicating whether the patient has been seen by a physician, seen by an advanced practice provider, together with the time spent by each provider, or patients needing to be seen, and a clinical indicator; generate a patient profile having the encounter indicator, an encounter time indicator, or both, including at least a portion of the received patient data, a summary of the patient's condition, and a follow-up task listing for display on the user device; and provide a user interface for modifying the patient profile to the user device over an encrypted network.

In still another embodiment, the system for processing and managing patient interactions includes: an activity log wherein additions, subtractions, modifications, edits, or all, related to a patient's record may be monitored and tracked. This allows both the ability to collect and input patient data but also to monitor time entries (e.g., logged time) of provider activity (see time function above) wherein a billing specialist may utilize the activity log to determine the current state of billing wherein a billing specialist is able to determine if a claim has been started, completed or if an active claim is pending (i.e., whether a claim has been generated and submitted for reimbursement).

Each preferred embodiment, taken individually or in combination with one or more other preferred embodiments, ultimately leads to overall increased efficiency in charge capture and claim generation wherein each implemented improvement provides for a feedback loop of fewer denied claims, greater number of accepted claims, increased accountability in claim submission, and more efficient and ergonomic structuring and display of provider code preferences - all creating new and novel efficiencies in charge claim capture. This improved charge claim capture system, informed by prior successful and “unsuccessful” claims submissions, results in ongoing, continual and enhanced advancements in ordered ranking of CPT and ICD codes, evidencing less waste of scare resources (provider and billing), ever accruing benefits of accurate billing procedures, monetary efficiencies and a more complete, precise and comprehensive system of healthcare delivery.

### BRIEF DESCRIPTION OF THE DRAWINGS

FIG. 1 illustrates a healthcare system network diagram depicting system components related to a healthcare charge capture and prioritization system, in accordance with one exemplary embodiment of the present disclosure;

FIG. 2 illustrates a schematic view of a healthcare charge capture and prioritization system, in accordance with one exemplary embodiment of the present disclosure;

FIG. 3 illustrates a view of user interface related to patient encounters, in accordance with one exemplary embodiment of the present disclosure;

FIG. 4 illustrates a view of a patient profile, in accordance with one exemplary embodiment of the present disclosure;

FIG. 5 illustrates a user interface related to provider charge capture, in accordance with one exemplary embodiment of the present disclosure;

FIG. 6 illustrates a flow chart diagram for prioritizing a Current Procedural Technology (CPT) code, in accordance with one exemplary embodiment of the present disclosure;

FIG. 7 illustrates a flow chart diagram for prioritizing an International Statistical Classification of Diseases and Related Health Problems (ICD) code, in accordance with one exemplary embodiment of the present disclosure;

FIG. 8 illustrates a flow chart diagram for assigning a prioritization ranking of an ICD code, in accordance with one exemplary embodiment of the present disclosure;

FIG. 9A illustrates a user interface listing CPT code favorites, ICD favorites, CLSL favorites;

FIG. 9B illustrates a user interface listing “favorite” CPT codes;

FIG. 9C illustrates a user interface listing “favorite” ICD codes;

FIG. 9D illustrates a user interface listing “favorite” CLSLs;

FIG. 10 illustrates a user interface incorporating a time capturing function;

FIG. 11 illustrates a user interface incorporating an enhanced Billing Module; and

FIG. 12 illustrates an exemplary activity log.

### DETAILED DESCRIPTION OF THE INVENTION

The preferred version of the disclosure presented in the following written description and the various features and advantageous details thereof, are explained more fully with reference to the non-limiting examples included in the accompanying drawings and as detailed in the description, which follows. Descriptions of well-known components have been omitted so as not to not unnecessarily obscure the principal features described herein. The examples used in the following description are intended to facilitate an understanding of the ways in which the disclosure can be implemented and practiced. Accordingly, these examples should not be construed as limiting the scope of the claims.

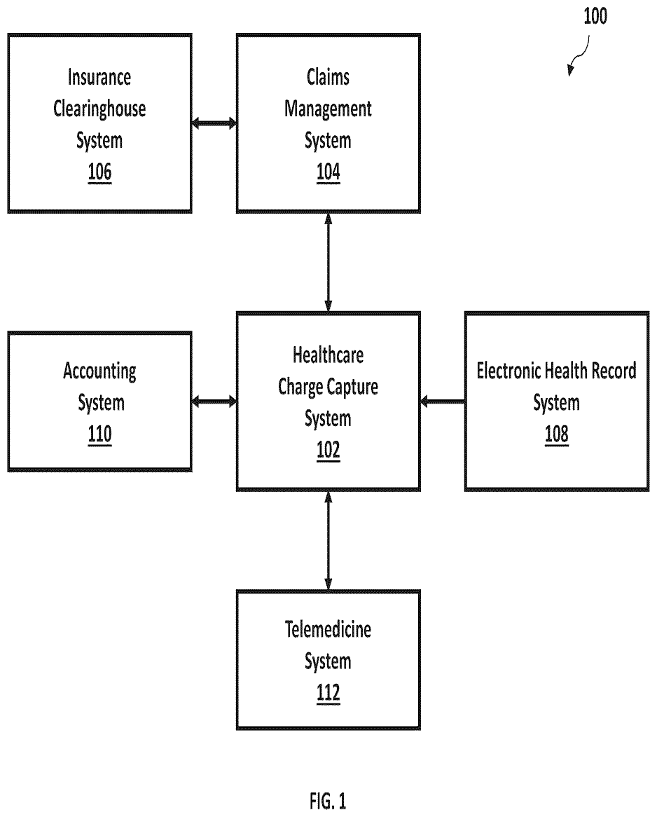

FIG. 1 illustrates a healthcare system network diagram 100 depicting system components related to a healthcare charge capture and prioritization system (CCS) 102, in accordance with one exemplary embodiment of the present disclosure. The healthcare system network 100 can include a healthcare charge capture system 102, a claims management system 104, an insurance clearinghouse system 106, an electronic health record system 108, an accounting system 110, and a telemedicine system 112.

The aforementioned system components 102, 104, 106, 108, 110, and 112 can be communicably coupled to each other via the Internet, intranet, or other suitable network. The communication can be encrypted, unencrypted, over a VPN tunnel, or other suitable communication means. The Internet can be a WAN, LAN, PAN, or other suitable network. The network communication between the system components 102, 104, 106, 108, 110, and 112, can be encrypted to ensure HIPAA compliance using POP, Blowfish, AES, 3DES, HTTPS, or other suitable encryption. The network communication can occur via application programming interface (API), health level 7 (HL7) standard, ANSI-X12, Ethernet, Wi-Fi, Bluetooth, or other suitable communication protocol. Additionally, third party databases can be operably connected to the system components 102, 104, 106, 108, 110, and 112.

The claims management system 104 can be a server or other suitable component configured to receive a claim from CCS 102 via open API or HL7. Healthcare claims management software can be used to streamline medical claims' processing and billing workflow. Healthcare claims management software can be sold either as standalone products or bundled within medical billing software, revenue cycle management software, or comprehensive medical practice management software. Payers, healthcare providers, and insurance providers typically use healthcare claims management system 104. The systems can be configured to either push or pull data between CCS 102 and the claims management system 104. The claims management system 104 can create an electronic file (i.e., a claim) known as an ANSI-X12 837 file. This file can be transferred to an insurance clearinghouse system 106.

Insurance clearinghouse system 106 can be a server or other suitable component operably coupled to claims management system 104 via the network. Insurance clearinghouse system 106 can function as an intermediary between a provider and a payer. The payer can be an insurance company, or other suitable entity. Insurance clearinghouse system 106 can review a submitted claim for errors, and finding none, can transmit the electronic claim over an encrypted network to the payer. The claim is then either approved or denied by the payer, in which an approved claim generates a payment of services to the provider, and a denied claim may require additional information to procure processing.

Electronic health record system (EHR) 108, a/k/a electronic medical record (EMR), can be a server or other suitable component configured to provide a combination of patient demographics, patient records, length of stay, chart review, insurance information, and order management, among others, to the CCS 102. This EHR/EMR platform integrates clinical and revenue cycle management systems to track invoices and payments via a patient account number or other suitable identifier. A patient can have a barcode on a wrist band, RFID, ID card, or other suitable indication of his or her account number. The CCS 102 can pull data from the EHR/EMR system 108 via API, HL7, or other suitable connections.

Accounting system 110 can be a server or other suitable component. Accounting system 110 can keep track of the cost per encounter, and other metrics related to the provider's services and billings.

Telemedicine system 112 can be a server or other suitable component providing patient related content to the provider, including streaming of audio, video, text, and other suitable content. Telemedicine system 112 can include a camera, microphone, or other suitable transducer to provide data to CCS 102.

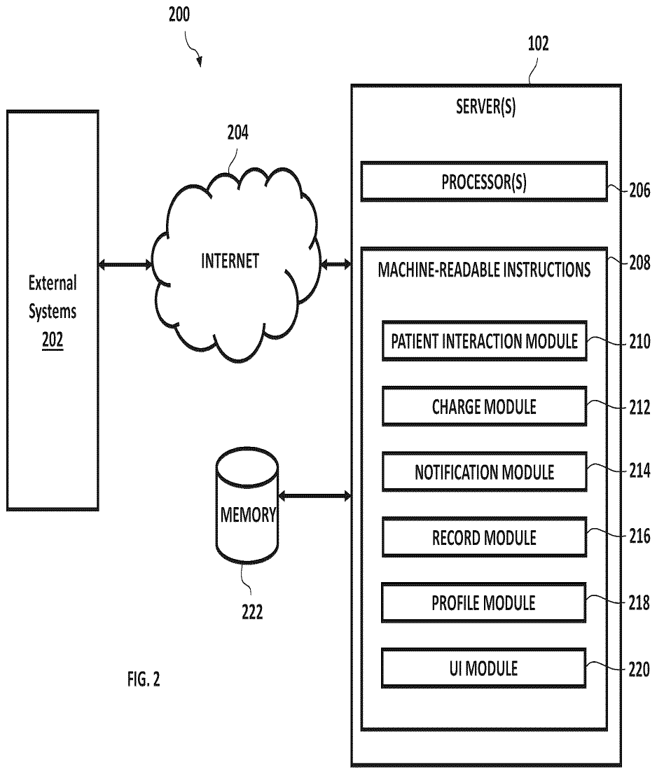

FIG. 2 illustrates a schematic view of a healthcare charge capture and prioritization system 102, in accordance with one exemplary embodiment of the present disclosure. Charge capture system 102 can include a server having one or more processors 206, a memory 222, machine readable instructions 208, including a patient interaction module 210, a charge module 212, a notification module 214, a record module 216, a profile module 218, and a UI module 220.

The CCS 102 can be communicably coupled to the Internet, intranet, or other suitable network(s). Such communication can be encrypted, unencrypted, over a VPN tunnel, or other suitable communication means. The Internet 204 can be a WAN, LAN, PAN, or other suitable network. The network communication between the system components 102, 104, 106, 108, 110, and 112, can be encrypted to ensure HIPAA compliance using PGP, Blowfish, AES, 3DES, HTTPS, or other suitable encryption. The network communication can occur via application programming interface (API), health level 7 (HL7) standard, ANSI-X12, Ethernet, Wi-Fi, Bluetooth, or other suitable communication protocol. Additionally, external systems, such as the system components 102, 104, 106, 108, 110, and 112, can be operably connected to the CCS 102 via the Internet 204.

The server can be implemented in hardware, software, or a suitable combination of hardware and software, and may comprise one or more software systems operating on one or more servers, having one or more processors 206, with access to memory 222. Server(s) can include electronic storage, one or more processors, and/or other components. Server(s) can include communication lines, or ports to enable the exchange of information with a network and/or other computing platforms. Server(s) can also include a plurality of hardware, software, and/or firmware components operating together to provide the functionality attributed herein to server(s). For example, server(s) can be implemented by a cloud of computing platforms operating together as server(s). Additionally, the server can include memory 222.

Memory 222 can comprise electronic storage that can include non-transitory storage media that electronically stores information. The electronic storage media of electronic storage may include one or both of system storage that is provided integrally (i.e., substantially non-removable) with server(s) and/or removable storage that is removably connectable to server(s) via, for example, a port (e.g., a USB port, a (rewire port, etc.) or a drive (e.g., a disk drive, etc.). Electronic storage may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage may include one or more virtual storage resources (e.g., cloud storage, a virtual private network, and/or other virtual storage resources). Electronic storage may store machine-readable instructions, software algorithms, information determined by processor(s), information received from server(s), information received from computing platform(s), and/or other information that enables server(s) to function as described herein. Electronic storage can also be accessible via a network connection.

Processor(s) may be configured to provide information processing capabilities in server(s). As such, processor(s) may include one or more of a digital processor, an analog processor, a digital circuit designed to process information, an analog circuit designed to process information, a state machine, and/or other mechanisms for electronically processing information, such as FPGAs or ASICs. The processor(s) may be a single entity or include a plurality of processing units. These processing units may be physically located within the same device, or processor(s) may represent processing functionality of a plurality of devices operating in coordination or software functionality.

The processor(s) can be configured to execute machine-readable instruction or learning modules by software, hardware, firmware, some combination of software, hardware, and/or firmware, and/or other mechanisms for configuring processing capabilities on processor(s). As used herein, the term “machine-readable instruction” may refer to any component or set of components that perform the functionality attributed to the machine-readable instruction component. This can include one or more physical processors during execution of processor readable instructions, the processor readable instructions, circuitry, hardware, storage media, or any other components.

The server can be configured with machine-readable instructions 208 having one or more functional modules. The machine-readable instructions 208 can be implemented on one or more servers, having one or more processors 206, with access to memory 222. The machine-readable instructions 208 can be a single networked node, or a machine cluster, which can include a distributed architecture of a plurality of networked nodes. The machine-readable instructions 208 can include control logic for implementing various functionality, as described in more detail below. The machine-readable instructions 208 can include a patient interaction module 210, a charge module 212, a notification module 214, a record module 216, a profile module 218, and a UI module 220.

The patient interaction module 210 can be configured to process and manage patient interactions and notifications, including receiving login credentials from a user device for a user over an encrypted network. The user (provider) can be a physician, an Advanced Practice Provider (APP) or other provider. The patient interaction module 210 can receive patient data for a plurality of patients at a first location from an electronic health record system for patients assigned to the user for a predetermined time period over an encrypted network. Patient data can include patient demographics, patient records, length of stay, chart review, insurance information, and order management, among others. The first location can be a hospital, unit, wing, floor, section, or other suitable location. A patient can be assigned to a provider indefinitely or for a predetermined period of time. The predetermined period of time can be periodic, such as a day, or aperiodic, such as the duration of a treatment. The patient interaction module 210 can determine a number of patient encounters for the user (provider) for the predetermined time period, time allotted to each patient (per each provider) and generate an indication of the number of patients seen, time seen, and unseen patients, and a progress bar representing a completion percentage of the number of patients seen for display on a user device. The indication can be provided to a user device such as a computer, server, mobile device, or other suitable device, for display. The patient interaction module 210 can generate a notification indicating those patients having outstanding encounters for display on the user device. The notification can include a portion of a patient profile, including patient information.

As discussed in more detail below, the charge module 212 can implement a plurality of algorithms to prioritize charge entries for a given encounter or procedure, including a Current Procedural Technology (CPT) code prioritization algorithm, an International Statistical Classification of Diseases and Related Health Problems (ICD) code prioritization algorithm, a time function, or a combination thereof. The algorithms and associated thresholds can be programmable to suit a particular provider, application, facility, or other requirement. The charge module 212 can correlate actual measured data with the calculated expected data to verify that the appropriate outcome is achieved. The measured data can include the time spent on each encounter/procedure, and time spent between each encounter/procedure, among others. This same time measurement may also inform the selection and utilization of certain claims having a time component where units of time may be different based on patients, providers, provider types, location, diagnosis and procedure(s). The expected data can include predetermined data stored in the memory, including the designated time for each encounter/procedure, and the designated time between each encounter/procedure, among others. The charge module 212 can also track analytics related to a user, including charge reporting, and number of completed encounters/procedures, the number of uncompleted encounters/procedures, time spent on completed encounters, or a combination thereof, among other measurables.

The notification module 214, can be configured to send and receive messages related to patient interaction(s) to and from the user device. The notification module 214 can generate one or more elements for display on the user device. The elements can provide additional information to the user related to patient encounters. For example, alerts can be generated by the notification module 214 and displayed on the user device, indicating completed encounters, outstanding encounters, a portion of the patient profile information, daily encounters entered or missing, encounter progress tracking, or other suitable information. Additionally, system symbols can be displayed on the user device to indicate task or patient status.

Advantageously, the record module 216 can generate a patient record for each patient using at least a portion of the patient data received by the server, including an encounter indicator, indicating whether the patient has been seen by a physician, seen by an advanced practice provider, or needs to be seen, and a clinical indicator. The encounter indicator can have a unique image, color, or other suitable symbol designating the particular status. The record module 216 can also include a user profile for the provider, indicating whether the provider is a physician, APP, another provider (e.g., respiratory therapist, phlebotomist), or administration staff. The user profile can also indicate patient listing, credentialing, specialization, open encounters, time spent on encounter, time-to-encounter completion, and other provider-related information.

The patient profile module 218 can generate a patient profile formatted for display in a user device. The patient profile is stored locally in memory 222, such that CPT and ICD codes for patient encounters can be associated with a patient, for ease of recall, monitoring, collection and analysis. The profile can include a listing of encounters, including an encounter summary, an in-patient follow-up, the date of service, times and duration of service, the location of the encounter, clinical indicators, the provider, the encounter indicator, at least a portion of the received patient data, a summary of the patient's condition, the status of billing, and a follow-up task listing for display on the user device among others. The patient profile can be a subset of the most relevant data from the patient record for a provider. However, the patient profile can be supplemented with additional relevant information not in the patient record.

The UI module 220 can provide a user interface for modifying the patient profile or patient record to the user device over an encrypted network. The UI module can generate data for presentation to the external systems 202 (user device) from any of the aforementioned modules 210, 212, 214, 216, 218, and 220. The UI module 220 can serve web pages or data, including via JavaScript, API, HTML, AJAX, RESTful services, or other suitable means. The UI module 220 can also provide a user interface for entering, reviewing or modifying the administrative portion of the CCS 102, including CPT and IDS codes ranking algorithms and thresholds, code preferences, time, billing and activity logs. The UI module 220 can additionally provide a user interface for generating user reports and viewing analytics related to any element of the CCS 102.

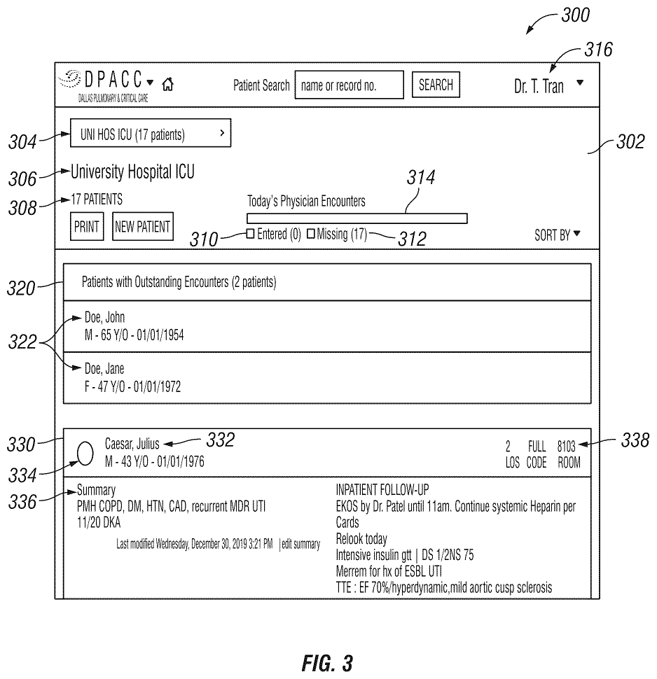

FIG. 3 illustrates a view of user interface 300 related to patient encounters, in accordance with one exemplary embodiment of the present disclosure. The patient encounter interface 300 can receive data from the UI module 220 to access and display data from any module of CCS 102, including the patient interaction module 210, profile module 218, and notification module 214. A first banner 302 is displayed across the top of the user interface. A user can access the system by providing valid credentials via a login, including a username and password, biometrics, multi-factor authentication, or other suitable credentialling. The name of the active user, as well as a menu 316 related to the user can be displayed at the top of the user interface. The first banner 302 can be configured to display data for a location 306 selected from a drop-down menu 304 related to the user. Only locations associated with the user via the user profile can be displayed.

Notifications of patients with outstanding encounters 320 can be displayed on the user interface 300. The notification can include patient information 322, including the patient's name, gender, age, birthdate, and other relevant patient information. At least a portion of the patient profile 330 can also be displayed on the user interface 300, along with a portion of the patient information 332, an encounter indicator 334, location-related information 338, and a summary of the patient's condition with a follow-up task listing 338 for display on the user device.

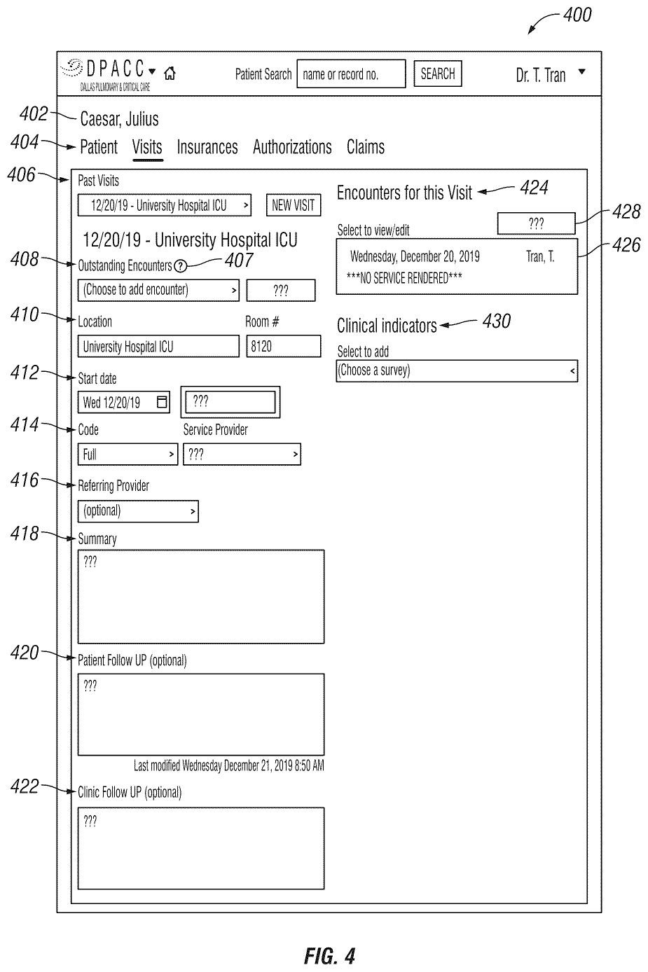

FIG. 4 illustrates a view of a patient profile 400, in accordance with one exemplary embodiment of the present disclosure. The patient profile 400 can receive data from the UI module 220 to access and display data from any module of CCS 102, including the patient interaction module 210, profile module 218, and notification module 214. The patient profile includes the patient name 402, patient-related navigation 404, and a user interface 406 for selecting, viewing, and modifying patient profile data. Navigation tools are provided to be able to review patient-related data for past visits, outstanding encounters 408, location 410, start date 412, code 414, referring provider 416, encounter summary 418, in-patient follow-up 420, clinic follow-up 422, encounters for this visit 424, encounter selection 426, add encounter 428, and clinical indicators 430.

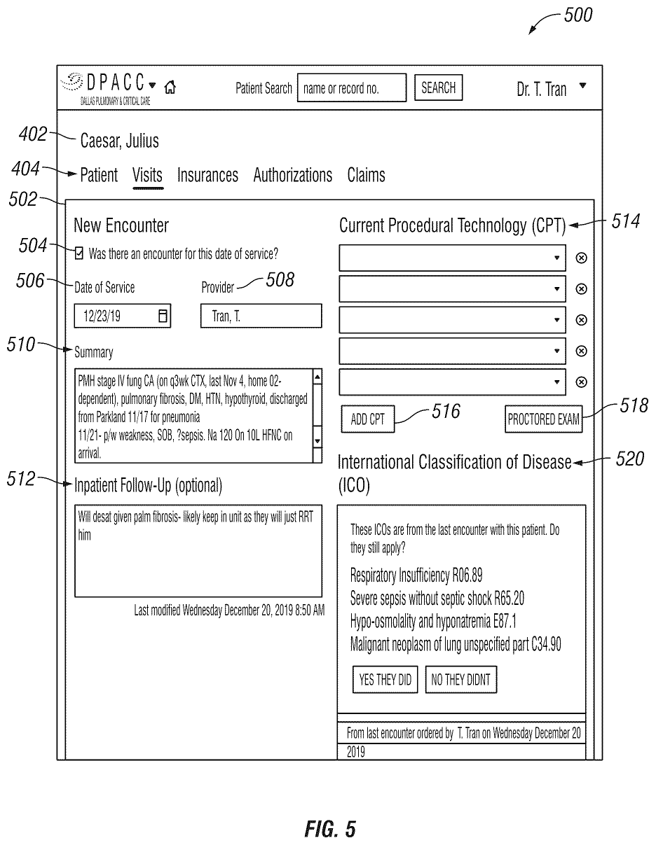

FIG. 5 illustrates a user interface related to provider charge capture 500, in accordance with one exemplary embodiment of the present disclosure. The charge capture user interface 500 can receive data from the UI module 220 to access and display data from any module of CCS 102, including the charge module 212, patient interaction module 210, profile module 218, and notification module 214. The provider charge capture interface 500 includes the patient name 402, patient-related navigation 404, and a user interface 502 for selecting, viewing, and modifying information related to a new patient encounter. The provider charge capture interface 500 further includes a selection element to indicate whether there was an encounter for a particular day of service 506, with the ability to designate a provider 508. Fields are provided to allow the user to enter an encounter summary 510 and in-patient follow-up 512. A CPT listing 514 indicates all CPTs for the encounter, with the ability to add an additional CPT 516 or add a proctored exam 518, wherein a user can identify which exam was proctored and by whom, on which date. An ICD listing 520 indicates all ICDs for the encounter, with the ability to add an additional ICDs. For ease of facilitation, the user is prompted whether ICDs from the last encounter with a patient still apply, such that this ICDs will be imported for the instant encounter, saving the provider from wasted time from entering repeat ICD codes. The charge capture user interface 500 can access the previous ICD codes via the profile module 218.

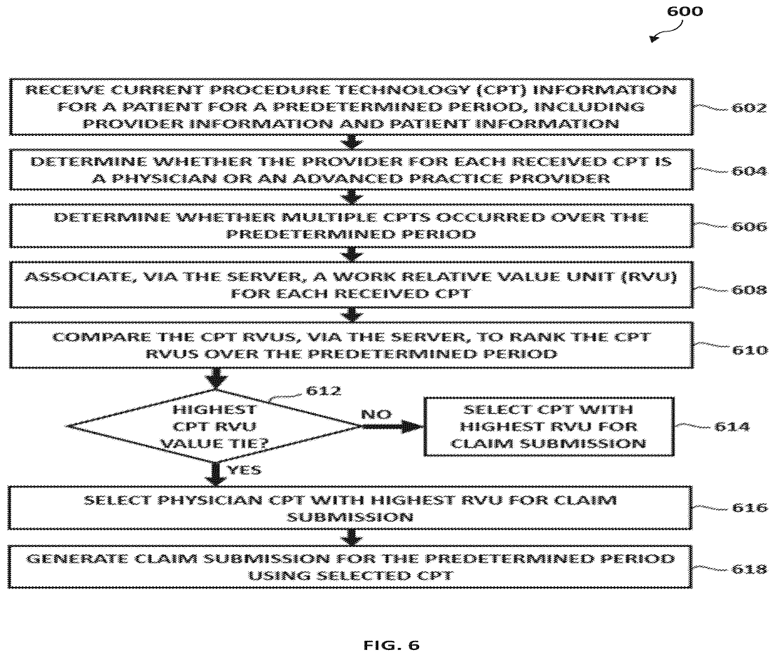

FIG. 6 illustrates a flow chart diagram 600 exemplifying control logic embodying features of a method for prioritizing a Current Procedural Technology (CPT) code, in accordance with one exemplary embodiment of the present disclosure. The CPT code prioritization control logic 600 can be implemented as an algorithm on a server, a machine learning module, or other suitable system. The CPT code prioritization control logic 600 can be achieved with software, hardware, an application programming interface (API), a network connection, a network transfer protocol, HTML, DHTML, JavaScript, Dojo, Ruby, Rails, other suitable applications, or a suitable combinations thereof.

The CPT code prioritization control logic 600 can leverage the ability of a computer platform to spawn multiple processes and threads by processing data simultaneously. The speed and efficiency of the CPT code prioritization control logic 600 is greatly improved by instantiating more than one process to generate a record having a patient's data. However, one skilled in the art of programming will appreciate that use of a single processing thread may also be utilized and is within the scope of the present invention.

The CPT code prioritization control logic 600 process flow of the present embodiment begins at step 602, where the control logic can receive Current Procedure Technology (CPT) information for a patient for a predetermined period, including provider information and patient information. The CPT information can be received from the CCS 102, memory 222, an EHR/EMR system 108, web portal, or other suitable mechanism. One or more CPTs can be received for one or more patients. The predetermined period of time is preferably a 24-hour period, but can be changed to any suitable period, as requested by the user. The CPT information can include CPT codes, patient information, profile information, or other suitable information. The control logic then proceeds to step 604.

At step 604, the CPT code prioritization control logic 600 can determine whether the provider for each received CPT is a Physician or an Advanced Practice Provider. The CPT code prioritization control logic 600 can access the provider profile to determine the type of provider, among other profile information, via an encrypted network connection, or other suitable means. The control logic then proceeds to step 606.

At step 606, the CPT code prioritization control logic 600 can determine whether multiple CPTs occurred over the predetermined period. If a single CPT occurred over the predetermined period, the control logic can select the single CPT for claim submission and proceed to step 618. However, should multiple CPTs be detected, the CPT code prioritization control logic 600 can search for Work Relative Value Unit (RVU) for each received CPT. For example, certain insurers or programs utilize a physician fee schedule to determine claim payments for provider services. The fee for each service can depend on RVUs, which rank the resources used to provide each service. These resources can include the physician's work, the expenses of the physician's practice, and professional liability insurance. The RVU can be generated and stored locally in a database in the memory 222, or can be accessed at an external system 202 via the Internet 204. The CPT RVU database can be accessed via a local area network, wide area network, application programming interface, or other suitable communication mechanism. The control logic then proceeds to step 608.

At step 608, the CPT code prioritization control logic 600 can associate, via the server, a Work Relative Value Unit (RVU) for each received CPT. The CPT code prioritization control logic 600 can edit the RVUs to account for certain factors associated with the CPT. Although physician CPTs typically have a greater RVU, given the type of procedure or the specialization, among other factors, the APP CPT may have a greater RVU. Any edits can be documented in the CCS 102 for auditing purposes. As each CPT has been assigned an RVU, the RVUs and associated CPTs can be ranked. The control logic then proceeds to step 610.

At step 610, the CPT code prioritization control logic 600 can compare the CPT RVUs, via the server, to rank the CPT RVUs over the predetermined period. The RVUs can be sorted according to sequential search, bubble search, or quick search algorithms, among others. The control logic then proceeds to step 612.

At step 612, the CPT code prioritization control logic 600 can determine whether there is a tie for the highest RVU among CPTs. From time-to-time, two or more CPTs can be assigned the same RVU. If there is a tie between two or more CPTs for the highest RVU, the control logic then proceeds to step 616. If there is not a tie between two or more CPTs for the highest RVU, the control logic then proceeds to step 616.

At step 614, the CPT code prioritization control logic 600 can select the CPT with highest RVU for claim submission. The selection can include flagging the selected CPT, storing the CPT in RAM, or otherwise designating the CPT as selected. The control logic then proceeds to step 618.

At step 616, the CPT code prioritization control logic 600 can select a physician CPT with the highest RVU for claim submission or APP CPT with the highest RVU. Typically, physician CPTs can be billed at a higher rate than APP CPTs, as such, the physician CPT may be prioritized over the APP CPT. Should no physician CPT exist among the tied CPTs, any APP CPT can be selected. Similarly, if only physician CPTs exist among the tied CPTs, any physician CPT can be selected. The selection can include flagging the selected CPT, storing the CPT in RAM, or otherwise designating the CPT as selected. The control logic then proceeds to step 618.

At step 618, the CPT code prioritization control logic 600 can generate, via the server, a claim submission for the predetermined period using the selected CPT. The claim can formatted in an ANSI-X12 837 file format, or other suitable format. The control logic then terminates or awaits a new CPT information and can repeat the aforementioned steps.

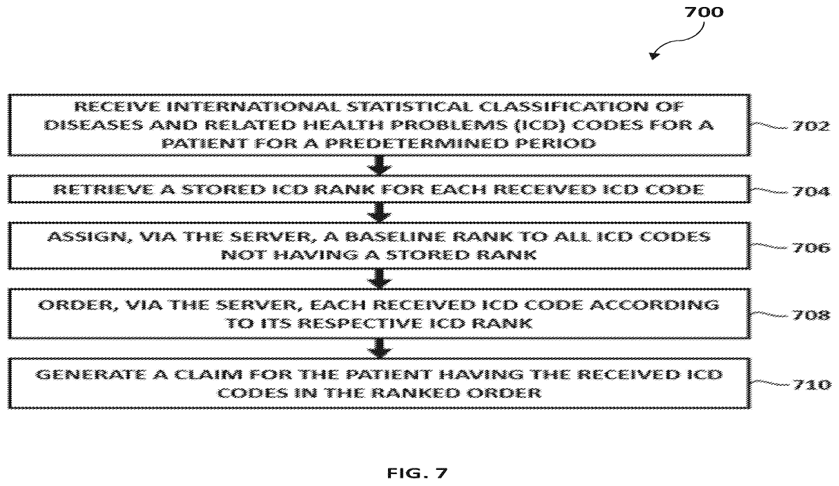

FIG. 7 illustrates a flow chart diagram 700 exemplifying control logic embodying features of a method for prioritizing an International Statistical Classification of Diseases and Related Health Problems (ICD) code, in accordance with one exemplary embodiment of the present disclosure. The ICD code prioritization control logic 700 can be implemented as an algorithm on a server, a machine learning module, or other suitable system. The ICD code prioritization control logic 700 can be achieved with software, hardware, an application programming interface (API), a network connection, a network transfer protocol, HTML, DHTML, JavaScript, Dojo, Ruby, Rails, other suitable applications, or a suitable combinations thereof.

The ICD code prioritization control logic 700 can leverage the ability of a computer platform to spawn multiple processes and threads by processing data simultaneously. The speed and efficiency of the ICD code prioritization control logic 700 is greatly improved by instantiating more than one process to generate a record having a patient's data. However, one skilled in the art of programming will appreciate that use of a single processing thread may also be utilized and is within the scope of the present invention.

The ICD code prioritization control logic 700 process flow of the present embodiment begins at step 702, where the control logic can receive International Statistical Classification of Diseases and Related Health Problems (ICD) information for a patient, for a predetermined period, including provider information and patient information. The ICD information can be received from the CCS 102, memory 222, an EHR/EMR system 108, web portal, or other suitable mechanism. One or more ICDs can be received for one or more patients. The predetermined period of time is preferably a 24-hour period, but can be changed to any suitable period, as requested by the user. The ICD information can include ICD codes, patient information, profile information, or other suitable information. The control logic then proceeds to step 704.

At step 704, the ICD code prioritization control logic 700 can retrieve a stored ICD rank for each received ICD code. If a particular ICD code was previously submitted in a claim using the CCS 102, the ICD code can be assigned a rank based upon whether the ICD code was accepted or denied. The ICD rank can be generated and stored locally in a database in the memory 222 or can be accessed at an external system 202 via the Internet 204. For example, the CCS 102 can generate an ICD rank for each ICD code submitted in a claim. In one exemplary embodiment, the rankings can range in value from 1 to 100. Alternatively, the rankings can range between 1 and 5, or any other suitable values. The ICD ranking database can be accessed via a local area network, wide area network, application programming interface, or other suitable communication mechanism. The control logic then proceeds to step 706.

At step 706, the ICD code prioritization control logic 700 can assign and store, via the server, a baseline rank for all ICD codes not having a stored rank. If an ICD code was not previously submitted by the CCS 102, it may not have a rank associated with the ICD code. ICD codes that are to be submitted for a first time can be assigned a baseline rank. In one exemplary embodiment, the baseline rank can be 50, however any value in the middle of the selected ranking range can be suitable as the baseline rank. The newly assigned rank can be stored in the ICD ranking database, or other suitable storage location. The control logic then proceeds to step 708.

At step 708, the ICD code prioritization control logic 700 can order, via the server, each received ICD code according to its respective ICD rank. ICD codes that were previously accepted will have a higher ranking than ICD codes that were previously rejected. As the order in which the ICD codes are presented to the payer matter, the ICD code prioritization control logic 700 can rearrange the ICD codes in a claim, placing the codes that are most likely to be accepted at the beginning of the list of ICD codes in the claim. The ICDs can be ordered according to sequential search, bubble search, or quick search algorithms, among others. The control logic then proceeds to step 710.

At step 710, the ICD code prioritization control logic 700 can generate a claim for the patient having the received ICD codes in the ranked order. The claim can be formatted in an ANSI-837 file format, or other suitable format. The control logic then terminates or awaits a new ICD information and can repeat the aforementioned steps.

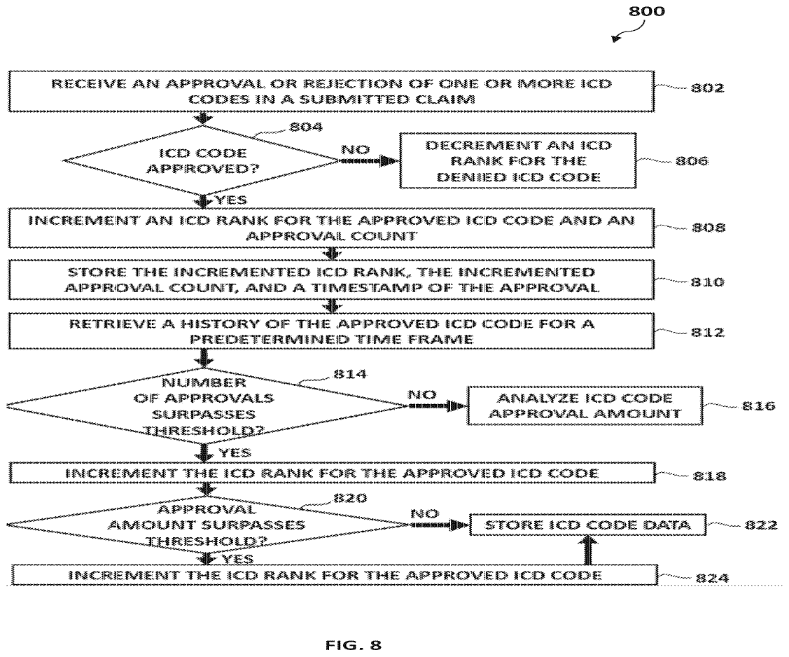

FIG. 8 illustrates a flow chart diagram 800 exemplifying control logic embodying features of a method for assigning a prioritization ranking of an ICD code, in accordance with one exemplary embodiment of the present disclosure. The ICD ranking control logic 800 can be implemented as an algorithm on a server, a machine learning module, or other suitable system. The ICD ranking control logic 800 can be achieved with software, hardware, an application programming interface (API), a network connection, a network transfer protocol, HTML, DHTML, JavaScript, Dojo, Ruby, Rails, other suitable applications, or a suitable combinations thereof.

The ICD ranking control logic 800 can leverage the ability of a computer platform to spawn multiple processes and threads by processing data simultaneously. The speed and efficiency of the ICD ranking control logic 800 is greatly improved by instantiating more than one process to generate a record having a patient's data. However, one skilled in the art of programming will appreciate that use of a single processing thread may also be utilized and is within the scope of the present invention.

The ICD ranking control logic 800 process flow of the present embodiment begins at step 802, where the control logic can receive an approval or rejection of one or more ICD codes in a submitted claim. After the CCS 102 submits a claim to the claims management system 104, the claims management system 104 can respond by providing an approval, partial approval, or a rejection of the claim. The control logic then proceeds to step 804.

At step 804, the ICD ranking control logic 800 can determine whether the submitted ICD code was approved or rejected. If the submitted ICD code was approved, the control logic then proceeds to step 808. If the submitted ICD code was denied, the control logic then proceeds to step 806.

At step 806, the ICD ranking control logic 800 can, via the server, decrement an ICD rank for a denied ICD code and increment a denial count for the denied ICD code. In one exemplary embodiment, the ICD ranking for a denied ICD code can be decremented by 10 units, however, any suitable decrement amount can be used. The denial count can be a counter or other suitable mechanism for storing the number of denials associated with the denied ICD code. The control logic then proceeds to step 810.

At step 808, the ICD ranking control logic 800 can, via the server, increment an ICD rank for the approved ICD code and increment an approval count for the approved ICD code. In one exemplary embodiment, the ICD ranking for an approved ICD code can be incremented by 10 units, however, any suitable increment amount can be used. The approval count can be a counter or other suitable mechanism for storing the number of approvals associated with the approved ICD code. The control logic then proceeds to step 810.

At step 810, the ICD ranking control logic 800 can store the incremented ICD rank, the incremented approval count, and a timestamp of the received approval. Additionally, the ICD ranking control logic 800 can store the decremented ICD rank, the incremented denial count, and a timestamp of the received denial. The control logic then proceeds to step 812.

At step 812, the ICD ranking control logic 800 can retrieve a history of the approved or denied ICD code for a predetermined time frame. The history can include the incremented/decremented ICD rank, the incremented approval/denial count, the payment amounts, and a timestamp of the received approval/denial, among other information. Certain conditions can be preferentially paid, if, for example, a certain number of approvals are granted for a particular ICD code within a shortened predetermined time frame or if the ICD code is paid out at a higher amount. The ICD code history can be utilized by the CCS 102 to analyze the metrics of the ICD code to determine whether to increment or decrement its ICD rank. The control logic then proceeds to step 814.

At step 814, the ICD ranking control logic 800 can determine whether the number of ICD code claim approvals surpasses a first threshold. The ICD ranking control logic 800 can compare, via the server, the number of ICD code claim approvals with the first threshold to determine whether the threshold is surpassed. The ICD ranking control logic 800 can include a feedback loop such that the thresholds can be modified using other information. For example, the ICD ranking control logic 800 can calculate the differences, standard deviation, average, interpolation, or other data analysis, on the information to set or modify the thresholds. If the number of ICD code claim approvals surpasses a first threshold, the control logic then proceeds to step 818. If the number of ICD code claim approvals does not surpass the first threshold, the control logic then proceeds to step 816.

At step 816, the ICD ranking control logic 800 can, via the server, analyze the ICD Code approval amount. Each ICD code can be approved for a certain approval amount, in dollars or other suitable currency unit. The ICD ranking control logic 800 can compare the ICD Code approval amount of the approved claim with the approval amounts stored in the ICD code history. Alternatively, the ICD ranking control logic 800 can decrement the ICD rank for the approved/denied ICD code, should the number of denials exceed a certain threshold within the shortened predetermined time frame. In one exemplary embodiment, the ICD ranking for a denied ICD code can be decremented by 10 units, however, any suitable decrement amount can be used. The control logic then proceeds to step 820. In another exemplary embodiment, the ICD ranking for a denied ICD code can remain unchanged.

At step 818, the ICD ranking control logic 800 can, via the server, increment an ICD rank for the approved ICD code and increment an approval count for the approved ICD code. In one exemplary embodiment, the ICD ranking for an approved ICD code can be incremented by 10 units, however, any suitable increment amount can be used. The approval count can be a counter or other suitable mechanism for storing the number of approvals associated with the approved ICD code. The control logic then proceeds to step 820.

At step 820, the ICD ranking control logic 800 can determine whether the ICD code claim approval amount surpasses a second threshold. The ICD ranking control logic 800 can compare, via the server, the claim approval amounts of the approved ICD code claim with the second threshold to determine whether the second threshold is surpassed. The ICD ranking control logic 800 can include a feedback loop such that the thresholds can be modified using other information. For example, the ICD ranking control logic 800 can calculate the differences, standard deviation, average, interpolation, or other data analysis, on the information to set or modify the thresholds. If the approval amount of the ICD code claim surpasses the second threshold, the control logic then proceeds to step 824. If the approval amount of the ICD code claim does not surpass the second threshold, the control logic then proceeds to step 822.

At step 824, the ICD ranking control logic 800 can, via the server, increment an ICD rank for the approved ICD code and increment an approval count for the approved ICD code. In one exemplary embodiment, the ICD ranking for an approved ICD code can be incremented by 10 units, however, any suitable increment amount can be used. The approval count can be a counter or other suitable mechanism for storing the number of approvals associated with the approved ICD code. The control logic then proceeds to step 820.

At step 822, the ICD ranking control logic 800 can store the ICD code data for the approved ICD code. The ranking, approval amounts, thresholds, and other relevant information can be stored in the ICD code history. The control logic then proceeds to step 820.

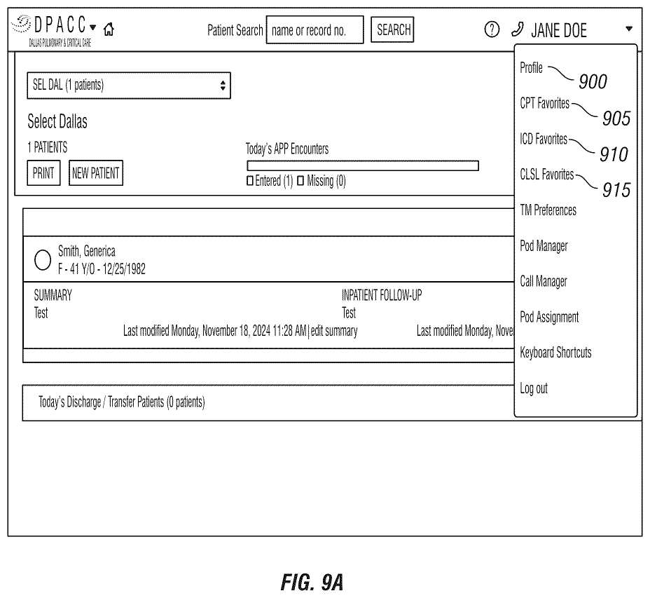

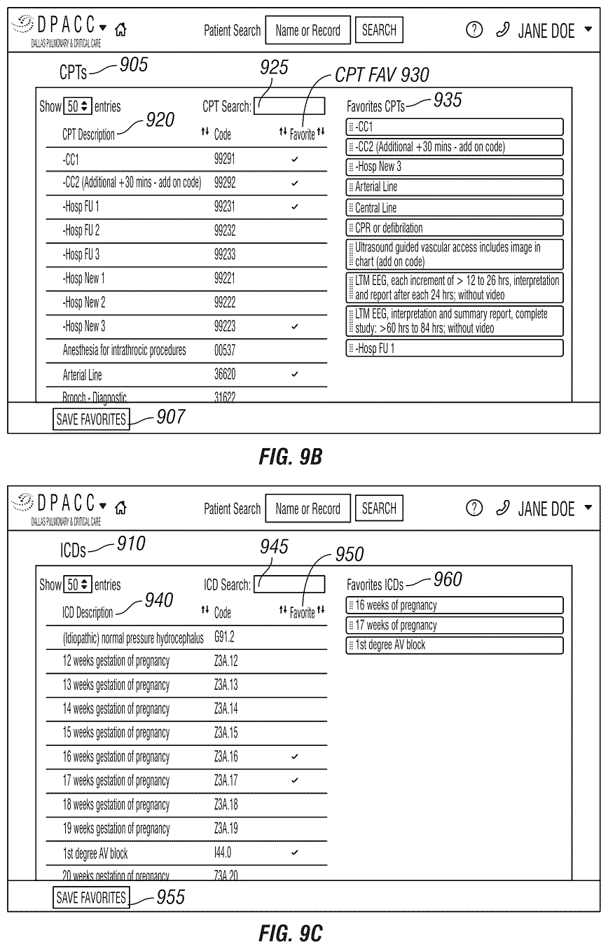

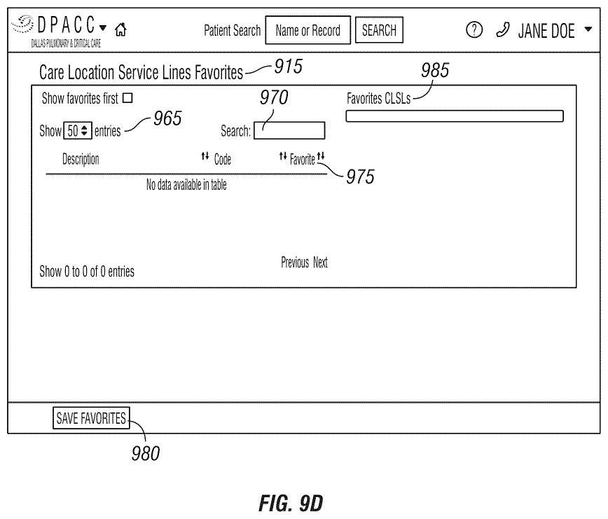

FIG. 9A-9D show an additional mechanism whereby once CPT codes, ICD codes, or both, are incremented, approval counts are incremented, or both, those codes may be ranked, ordered and preferential displayed to a provider's “favorites” for expediated access and use. This ranking and ordering may be achieved through a control logic wherein a server can be configured with machine-readable instructions 208 having one or more functional modules for prioritizing CPT codes, ICD codes, or both. The machine-readable instructions 208 may be implemented on one or more servers, having one or more processors 206, with access to memory 222. The machine-readable instructions 208 can be a single networked node, or a machine cluster, which can include a distributed architecture of a plurality of networked nodes. The machine-readable instructions 208 can include control logic for prioritizing CPT codes (see FIG. 6), ICD codes (See FIGS. 7-8), or both is achieved.

As provided, incremented CPT codes, ICD codes, or both, may be prioritized through control logic 600 via software, hardware, an application programming interface (API), a network connection, a network transfer protocol, HTML, DHTML, JavaScript, Dojo, Ruby, Rails, other suitable applications, or a suitable combination thereof.

Control logic, as described, can leverage the ability of a computer platform to spawn multiple processes and threads by processing data simultaneously. The speed and efficiency of the CPT and/or ICD code prioritization control logic 600 is enhanced by instantiating more than one process to generate a data set of prioritized CPT and/or ICD codes. However, one skilled in the art of programming will appreciate that use of a single processing thread may also be utilized and is within the scope of the present invention.

Of note, the prioritization and display of CPT and ICD code “preferences”, “favorites”, or prioritized codes may be ranked and ordered based on one or more identifiable criteria including, but not exclusive of, specific provider proclivity and use, provider type (Physician, APP or other medical provider), incremented claim approval(s), incremented claim count approval, incremented claim amount approval, care location, facility type, thresholds, or a combination thereof.

In opposite, de-prioritization, de-listing or removal of CPT and ICD code “preferences”, “favorites”, or prioritized codes may be ranked and ordered in the negative based on one or more identifiable criteria including, but not exclusive of, specific provider non-use, exclusion based on provider type, decremented claim approval(s), decremented claim count, decremented claim amount, exceeding the perimeters of a care location, incorrect facility, failure to meet thresholds, or a combination thereof.

Furthermore, prioritizing and deprioritizing may be based on one or more set or adjustable thresholds wherein, if a threshold is met or surpassed, those CPT codes and ICD codes meeting or exceeding the threshold may be prioritized and those failing to exceed the threshold may be de-prioritized and/or delisted from favorites. Too the threshold may be implemented in the negative wherein those CPT codes and ICD codes which fail to meet or fall below a threshold may be decremented, lowered in order or rank or excluded from a provider's favorites.

Moreover, these preferences may be manually added or subtracted by a provider, auto populated (or de-populated) by a platform administrator or algorithmically through control logic, or a combination thereof, wherein the preference ranking and ordering of CPT and ICD codes may be amenable to a feedback loop which may be performed by control logic whereby prioritizations and de-prioritizations are implemented on a continuous basis, based on a criterion or criteria, wherein control logic can calculate the differences, standard deviation, average, interpolation, or other data analysis, on the information to set or modify a threshold or thresholds and thereby cause the incrementing, decrementing, inclusion or exclusion from a provider's favorites list.

As illustrated in FIG. 9A, a provider (or other user) may access provider favorites via a UI and dropdown menu wherein a selectable profile 900 (here of patient “Jane Doe”) is accessible as well as “favorites” listings CPT Favorites 905, ICD Favorites 910 and CLSL (Care Location Service Lines) Favorites 915) wherein is CPT Favorites 905 is selectable to CPT Favorites in FIG. 9B, ICD favorites 910 is selectable to FIG. 9C, and CLSL Favorites is selectable to FIG. 9D displayed and selectable through user interface in FIG. 9A for selection by the provider, an administrative user, or both.

As shown in FIG. 9b, prioritized and/or preferred ‘CPT Favorites’ 905 are accessible via a UI and provider selection of CPT favorites 905 (see FIG. 9A) via search function 925 providing of CPT code descriptions 920 where CPT codes are further subdivided and selectable as those CPT code favorites 930 wherein the user (e.g., a provider, administrator or other user) selects and assigns CPT codes manually to the favorites list (Favorites CPTs 935) and then saves “favorites” via save favorites 907 for inclusion into favorite CPTs 935 as prioritized, preference or favorite CPT codes. This selection may further be simply in relation to the specific patient being treated, in relation to the facility, specific to a provider (or provider type) which is selected by a user, algorithmically derived (via control logic) based on use, an individual provider, provider type, location, facility, or various selection criteria (as described above) or based on provider proclivity, or both. These Favorite CPT codes 935 may include a truncated subset of all available CPT codes or a modified and prioritized subset thereof which is modifiable based on control logic, system administrator, or provider selections and/or prioritization. What is more, prioritized CPT codes may be dynamic in response to changes in criteria or through the utilization of control logic, provider selection or a third-party charge capture administrator (i.e., billing specialist). And, as each provider (or administration specialist), by selecting CPT codes, informs the ongoing and perpetual list of preferred CPT codes selected (by previously described control logic) or selectable (by provider or third party), or both, via the aforementioned feedback loop, where CPT codes may be revised, updated, incremented, decremented, prioritized and de-prioritized, included or excluded, through ongoing control logic, provider selection, or an administrative function, or a combination thereof, algorithmically based on the criteria including, but not exclusive of, specific provider proclivity and use, provider type (Physician, APP or other medical provider), specialty area (e.g., urgent care, cardiology, pulmonary care), charge capture administrators, through incremented claim approval(s), incremented claim count, by care location, a threshold or thresholds, or any combination thereof. In addition, criteria, where two or more criteria are selected, may be equally weighted or assigned a percentage weight base where each criterion may have unequal weight in the determination of prioritization. This is further modifiable via a feedback loop wherein past, historic, prior or previous selections may be analyzed, via control logic, to inform the selection (and de-selection) of CPT code for inclusion in (and exclusion from) Favorite CPTs 935.

Similarly, FIG. 9C, ICDs 910, displays a description of ICD codes 940 whereby ICD codes are searchable 945 to provide a list of ICD codes from which favorites are selectable and savable 955 as ICD favorites 950 to generate a subset of ICD codes (favorite ICDs 960) which are further subdivided and selectable as those ICD codes which the provider is currently assigning to the favorites list, generally, or simply in relation to the specific patient being treated. And, as each provider, by selecting ICD codes 910 informs the ongoing list of segregated ICD codes selected (by control logic) or selectable (by provider or a system administrator), or both, via the aforementioned feedback loop where ICD codes may be incremented, decremented, prioritized and de-prioritized, included and excluded through ongoing control logic, provider selection or both, algorithmically, based on the criteria of specific provider proclivity and use, provider type (Physician, APP or other medical provider), specialty area (e.g., urgent care, cardiology, pulmonary care), incremented claim approval(s), incremented claim count, care location, threshold(s), or any weighted averages assigned or assignable or a combination thereof. Yet, it is to be understood that selection of ICD favorites may be based on corresponding criteria to CPT selection, based on a one or more of the criteria for CPT selection, either manually or algorithmically (through control logic) or a combination thereof.

Similarly, FIG. 9D illustrates Care Location Service Lines (CLSL) Favorites 915, displaying a list of CLSL descriptions 965 from which favorites 975 are selectable and savable 980 thus creating a subset of Favorite CLSLs 985 which are further subdivided and selectable as those CLSLs, which the provider is currently assigned to or is assigned to a favorites list 985, generally, or simply in relation to the specific patient being treated and further based on a specific treatment location, including telemedicine and virtual appoints (which may be integrated into a an International Statistical Classification of Diseases and Related Health Problems (ICD) and Current Procedural Technology (CPT) code prioritization system as related to a selected or indicated CPT code indicated by telemedicine encounter 1010). And, as each provider, by selecting CLSLs, or credentialed or privileged in a particular setting, hospital or facility, informs the ongoing set of segregated CLSLs selected (by logic) or selectable (by provider or a system administrator), or determined by provider type, practice or privileges, via the aforementioned feedback loop where CLSLs may be revised, updated, included, excluded, incremented, decremented, prioritized and de-prioritized through ongoing logic, provider selection or both, algorithmically, based on the criteria of specific provider location, practice location, practice region, provider type (Physician, APP or other medical provider), specialty area (e.g., urgent care, cardiology, pulmonary care), incremented claim approval(s), incremented claim count in specific facility or care location, threshold(s), or any weighted averages or a combination thereof.

In terms of a threshold or thresholds, thresholds may be modified via control logic which can calculate the differences, standard deviation, average, interpolation, or other data analysis, on the information to set or modify the thresholds. Thresholds may also be determined and set manually by a provider, system administrator or other user either with or without analyzed data generated through ongoing use of control logic.

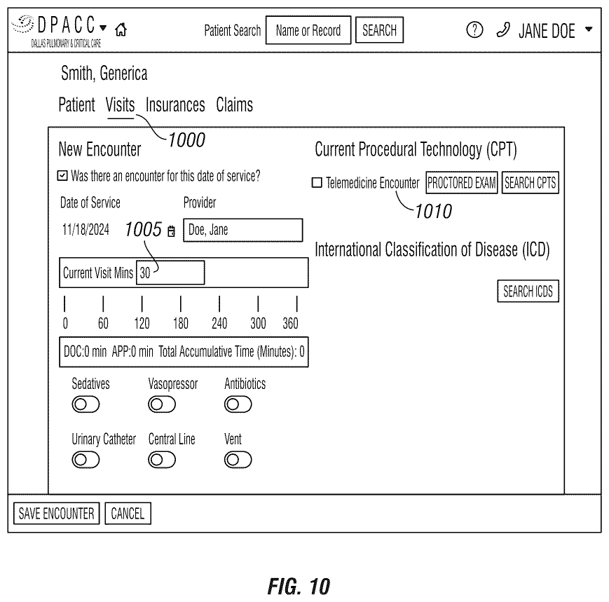

FIG. 10 illustrates a determination of patient visits 1000 including both date of service and, most applicable to the present invention, time spent in a patient-physician encounter represented by current visit minutes 1005. Time, in units of minutes or fractions of an hour, may be captured on a provider-by-provider basis where time is divided between a physician provider (MD or DO) and an advanced practice provider (APP) ensuring the billing system captures a claim or claims wherein the value of time between and among providers is typically disparate. This allows for accurate accounting of those providers expending the largest time and billable rate to an appropriate CPT code ensuring the inclusion of appropriate modifiers, when available, are included per billing guidelines when multiple providers have provided care to the same patient on the same date of service or at the same time.

This is especially important to account for time where the Centers for Medicare and Medicaid Services (CMS) permits split or shared Evaluation and Management (E/M) visits such as critical care services, emergency care services, and many other services, so long as a patient visit occurs in the appropriate settings and services are rendered by the appropriate providers, taking into account time spent on the encounter and/or level of care.

Customarily, split or shared services are performed jointly by a physician and a Non-physician Practitioner (NPP) such as a Nurse Practitioner or Physician Assistant where both providers render services to a patient at the same or different times during the same calendar day and combine their services so that whomever spends more than 50% of the combined time or performs and documents the majority of the medical decision-making, is indicated as the primary caregiver creating the basis of the submitted claim for reimbursement. For example, the Centers for Medicare and Medicaid Services (CMS) may reimburse 100% of the Physician Fee Schedule if the physician bills and only 85% of the fee schedule if the NPP also bills. In order to capture split or shared billing, the present Charge Capture system has been equipped with an integratable time function feature with which to capture this time.

FIG. 10 illustrates a UI when a provider accesses this UI, in this instance a physician or an Advanced Practice Provider (APP), enters an encounter for a specific date of service, there is a time indicator which defaults to 30 minutes (for current visit minutes 1005). The minimum time of 30 minutes is set as a default due to the minimum time threshold for the initial 99291 critical care code being set at 30 minutes; this ensures that the time threshold is achieved as per critical care billing guidelines. In the present instance, the physician and APP enter separate encounters and times for the same date of service whereby their total time is then aggregated. As to account for each provider's time, an accumulative time tracking bar may display how much time is entered individually by each provider as well as the total aggregated time for a date or dates of service. The system's control logic is then capable of recognizing each providers'time entries, separable and in sum, and thus submit a claim under the provider who entered the most time or under the time affording the highest reimbursement rates. If the physician's time (i.e. billable rate) is equal or greater than the APP's, the system will choose the physician's encounter to be submitted once claims are ready to be sent to the practice management system and to insurance companies. The aggregated time box will also prompt the providers, that if total combined time is at least 104 minutes, the billing provider may bill for the initial 99291 code with the additional add-on 99292 critical care code. This enhancement ensures billing reimbursement is optimized.

And, although CPT codes for Critical Care codes 99291 and 99292, for example, do not have a specific time attached to them per se, these codes do have a time component (time thresholds) that must be met in order to bill for those CPT codes. For instance, if a provider bills a 99291 code, it is assumed the provider spent at least 30 mins on the visit but, the provider could have spent 30-74 mins, therefore for this purpose, where the time function is calculated separately the time influences the reimbursement rate as a factor for claim billing purposes. Therefore, CPT code often carries with them an integrated time element which must be accounted for in the billing of claims, but also must be considered where minimum and maximum times influence time spent by a provider on delivery of patient care.

In the present invention time is further identifiable as a factor for appropriate reimbursement where if the time threshold or total combined/shared Critical Care time is met (in this instance 104 mins and subsequent increments of 30 mins thereafter), that the system will prompt/flag the provider that additional critical care time of 99292 may be billed which this may also be automated, where a charge capture system (CCS) may flag, or otherwise alert the provider and/or billing specialist, of time allotted as well as other keywords that may be documented in the providers' notes audited directly from the EHR through ADT integration and thereby ensure a higher probability of claim acceptance and/or claim accuracy in billing by the selection of both the proper CPT (and related ICD code) code but also validating that the appropriate time component of these CPT codes has been properly documented and that all allotted time has been accounted for.

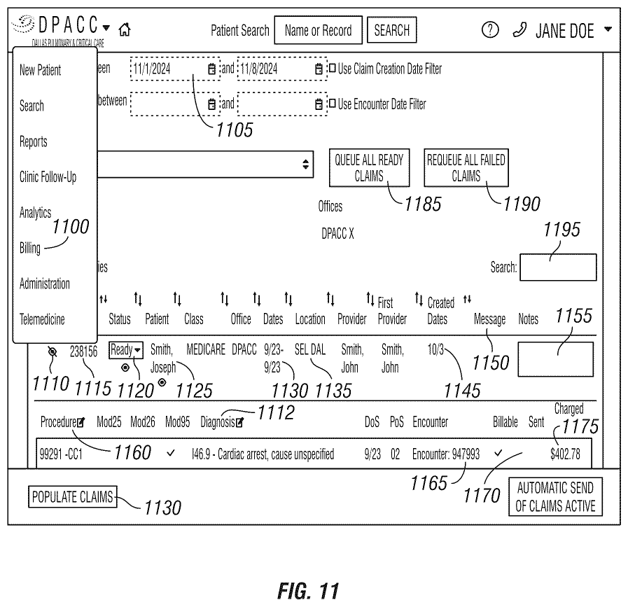

FIG. 11 illustrates, within a representation of a Billing Module 1100, accessible via an interface (UI) whereby control logic, billing specialists, or a combination thereof, populate claims created by providers after an encounter with a patient which is submitted within a specific care location for a given encounter time and duration. The claims themselves may be populated and filtered by a specified date range 1105 and further defined by patient (Jane Doe), procedure 1160 and/or provider 1125. The billing specialists are thus able to filter claims that have been sent or are ready to be submitted 1120 to the revenue cycle management as well as claims that have failed for reasons such as missing demographics or insurance information based on error messages.

As in FIG. 11, a user interface action label is actuatable to access a Billing Module 1100 wherein the user is able to search claims and encounters by a specific date range 1105. The user may then toggle to visualize additional hidden information, specifically the CPTs/procedures, modifiers (e.g., time), and/or ICD codes reported by the provider 1110. Other information included on the Billing Module 1100 interface includes claim number 1115 designated for tracking purposes, an indicator for posting status of the claim (i.e., whether a claim has been sent, is ready to be sent to revenue cycle management system), or if the claim failed to transmit 1120, financial class of the patient 1125 (i.e., Medicare), date(s) of service 1130, provide name 1122, date of claim creation 1145, message failure to send 1150, a note taking feature 1155 (for encounter and/or billing information), code insertion and deletion 1160, encounter number 1165 for tracking purposes, a “check” 1170 if a claim was submitted, a list of charges, corresponding with input of fee schedule for particular procedure 1175, a user interface action button to manually populate claims 1180, a user interface action button to queue all “ready” claims to be sent to revenue cycle management system 1185, and a user interface action button to requeue all failed claims to be sent to revenue cycle management system 1190 as well as a search function 1195 and the ability to select automatic sending of active claims 1199.

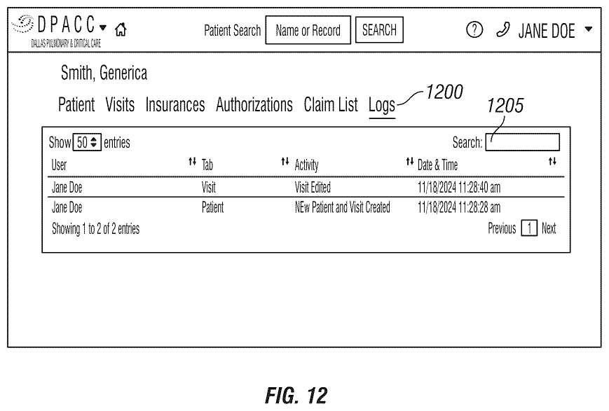

Too, FIG. 12 displays an activity log 1200 whereby log history and prover activity may be entered, edited, and date and time stamped for increased accuracy and accountability where action label 1200 allows for access to an activity module and a log search function 1205 which may be assignable to a specific patient and user for data entry and accounting which may be pertinent to the selection of ICD codes, CPT codes, procedure identification, location verification and identification and tracking of time functions.

These two latter functions, billing and activity log, have primarily accounting and diagnostic verification functions, which, while serving a primarily derivative purpose, form critical system components of confirming and verifying data input on which to base the final steps of charge capture - ensuring proper claim submission for reimbursement as they related to diagnosis ICD codes, CPT and CPT RVU use and code selections, time tracking, and split time encounters, among others.

The present invention and method of use therefore provides at least the following advantages: (1) an integration of a claims management system (and related insurance clearinghouse system), an accounting system, an electronic health record system and a telemedicine system into an improved charge capture system forming the nexus and basis of all interconnected systems, (2) ranking, ordering, incrementing and decrementing provider selected Current Procedural Technology (CPT) and International Statistical Classification of Diseases and Related Health Problems (ICD) codes through prioritization algorithms and control logic to increase the probability of claim acceptance, reduce the overutilization of processing resources and network inefficiencies from repeated system denials, (3) assuring the highest reimbursement rate based on diagnosis (ICD code) as it relates to CPT RVUs between and among physicians and APPs, (4) ranking, ordering and incrementing (and/or decrementing) CPT codes, ICD codes and CLSL preferences, saved as “favorites”, through prioritization (and de-prioritization) via a provider, administrator or other user, algorithms based on criteria including, but not exclusive to, applicability, provider, provider type, indication, use and claim acceptance and reimbursement for ergonomic data capture, improved accuracy and conservation of scare resources, (5) creating an analytical framework for feedback loops of increasingly quality of pertinent data to constantly inform, update and revise the prioritization of CPT and ICD codes, facility codes and CPT and ICD code selectable preferences by a provider or system administrator, (6) incorporating a time function into charge capture as to ensure proper procedure accounting between and among providers for proper reimbursement, (6) more timely and accurate billing procedures, and (7) improved activity log accountability.

Persons skilled in the art will readily understand that these advantages (as well as the advantages indicated in the summary) and objectives of this system would not be possible without the particular combination of computer hardware and other structural components and mechanisms assembled in this inventive system and described herein. It will be further understood that a variety of programming tools, known to persons skilled in the art, are available for implementing the control of the features and operations described in the foregoing material. Moreover, the particular choice of programming tool(s) may be governed by the specific objectives and constraints placed on the implementation plan selected for realizing the concepts set forth herein and in the appended claims.

The disclosure may be embodied in other specific forms without departing from the spirit or essential characteristics thereof. For example, each of the new structures described herein, may be modified to suit particular local variations or requirements while retaining their basic configurations or structural relationships with each other or while performing the same or similar functions described herein. The present embodiments are therefore to be considered in all respects as illustrative and not restrictive. Accordingly, the scope of the inventions are established by the appended claims rather than by the foregoing description. All changes which come within the meaning and range of equivalency of the claims are therefore intended to be embraced therein. Further, the individual elements of the claims are not well-understood, routine, or conventional. Instead, the claims are directed to the unconventional inventive concept described in the specification.

We claim:

1. An International Statistical Classification of Diseases and Related Health Problems (ICD) preferred code prioritization system, comprising:
a server having a system processor running a healthcare charge module configured to, in order to process and prioritize disease and related health problem codes, order and rank ICD codes by:
receiving an approval or rejection of one or more ICD codes related to one or more procedures, diseases, related health problems, or a combination thereof, in a submitted claim;
incrementing an ICD code rank for the approved ICD code, incrementing an approval count, if an ICD code is approved, or both;
decrementing the ICD code rank for the rejected ICD code and incrementing a rejection count, if an ICD code is rejected;
storing the ICD code rank, the incremented approval count, the rejection decremented count, and a timestamp of the approval or disapproval for the received approval or rejection;
creating a baseline for ICD codes not having a stored rank;
retrieving a history of approved ICD codes, rejected ICD codes, or both, for a predetermined time frame;
ordering and ranking each ICD code;
incrementing the ICD code rank for the approved ICD code, otherwise, analyzing the ICD code approval amount, if a number of approved ICD codes surpasses a first threshold;
incrementing and storing the ICD code order and rank for an approved ICD code, decrementing the ICD code rank for a rejected ICD code;
otherwise, storing the ICD code rank, if the ICD code approval amount surpasses a second threshold;
decrementing and storing the ICD code rank for a rejected ICD code, otherwise, storing the ICD code rank, if the ICD code is approved and under said second threshold or rejected;
incrementing an ICD code rank for an approved ICD code and incrementing an approval count for the number of approved ICD codes;
ordering and ranking ICD codes, incremented ICD codes, ranked ICD codes, ICD approval counts, ICD code approval amount, rejected ICD codes, ICD rejection amount, or a combination thereof; and
listing ICD codes prioritized highest to lowest increment, ranked order, or a combination thereof, for prioritized, preferenced or incremented codes for provider selection or selections.

2. The International Statistical Classification of Diseases and Related Health Problems (ICD) code prioritization system of claim 1, wherein prioritized, preferenced or incremented code provider selection or selections, is based on provider manual selections, provider use, provider type, care location, location type, claim acceptance, claim count, claim amount, reimbursement rates, thresholds, or a combination thereof.

3. The International Statistical Classification of Diseases and Related Health Problems (ICD) code prioritization system of claim 2 wherein if an ICD code is approved, an ICD rank is incremented for the approved ICD code and an approval count is tallied and incremented, if an ICD code is denied, the ICD rank is decremented for the denied ICD code and a denial count is tallied and decremented which is then stored to a memory.

4. The International Statistical Classification of Diseases and Related Health Problems (ICD) code prioritization system of claim 3, wherein ICD code prioritization control logic is implemented as an algorithm on a server, a machine learning module, or other suitable system and achieved with software, hardware, an application programming interface (API), a network connection, a network transfer protocol, HTML, DHTML, JavaScript, Dojo, Ruby, Rails, other applications, or a combination thereof.

5. The International Statistical Classification of Diseases and Related Health Problems (ICD) code prioritization system of claim 4, wherein said control logic leverages the ability of a computer platform to utilize a single processing thread or spawn multiple processes and threads by processing data simultaneously through two to a plurality of processes for two to a plurality of patients to order and rank ICD codes.

6. The International Statistical Classification of Diseases and Related Health Problems (ICD) code prioritization system of claim 1, wherein said predetermined timeframe may be periodic or aperiodic.

7. The International Statistical Classification of Diseases and Related Health Problems (ICD) code prioritization system of claim 5, wherein those ICD codes not having a stored rank or not previously being submitted said system's control logic are assigned said baseline rank.

8. The International Statistical Classification of Diseases and Related Health Problems (ICD) code prioritization system of claim 7, wherein stored ranked ICD codes and non-stored, non-ranked ICD codes are arranged and ranked by said control logic according to those most likely to be accepted by a payor based on ICD code rank, ICD code incremented count, ICD code approval count, ICD approval amount, or a combination thereof.

9. The International Statistical Classification of Diseases and Related Health Problems (ICD) code prioritization system of claim 8, wherein control logic ranked ICD code payment amounts are prioritized as charge entries for a given encounter or procedure whereby ICD prioritization are determined algorithmically to determine efficiency metrics and payor reimbursements.

10. The International Statistical Classification of Diseases and Related Health Problems (ICD) code prioritization system of claim 2, wherein ICD codes are selected from a larger code set based on provider use, provider type, care location, location type, incremented codes, reimbursement rates, or a combination thereof, based on one or a number of factors including utilization, provider specialty field, a specific provider, a provider type, congruence with a billing module, provider population, billing specialists population, auto-population, provider location, algorithmically selected based on accepted or rejected claims, and thresholds, for expedience and ease of access.

11. An International Statistical Classification of Diseases and Related Health Problems (ICD) and Current Procedural Technology (CPT) code prioritization system, wherein control logic orders and ranks ICD codes according to historical ICD code acceptance, ICD code acceptance rates, accepted CPT codes and accepted CPT RVU values giving the highest probability of acceptance at the highest reimbursement rates by the following:
associating an ICD code or ICD codes with a patient diagnosis;
associating a CPT code or codes with said ICD code;
receiving CPT information for said patient for a predetermined period, including provider information and patient information;
determining whether the provider for each received CPT is a physician or an advanced practice provider (APP);
determining whether multiple CPTs occurred over the predetermined period;
associating, via the server, a work relative value unit (RVU) with each received CPT;
comparing the CPT RVUs, via the server, to rank the CPT RVUs over the predetermined period;
generating a CPT RVU database;
selecting a physician CPT with the highest RVU for claim submission, if there is a tie for the highest CPT RVU value, otherwise, selecting a CPT with the highest RVU for claim submission;
ranking and ordering CPT codes; and
listing CPT codes prioritized highest to lowest increment, ranked order, or a combination thereof, for prioritized, preferenced or incremented codes for provider selection or selections.

12. The International Statistical Classification of Diseases and Related Health Problems (ICD) and Current Procedural Technology (CPT) code prioritization system code prioritization system of claim 11, wherein, if two to a plurality of CPTs are detected over a predetermined period, said control logic operates by:
searching for work relative value units (RVUs) for each CPT to create a CPT RVU database for CPT code prioritization.

13. The International Statistical Classification of Diseases and Related Health Problems (ICD) and Current Procedural Technology (CPT) code prioritization system code prioritization system of claim 12, wherein fee for each service depends on RVUs and RVUs are calculated based on resources including: physician's work, the expenses of the physician's practice, and professional liability insurance.

14. The International Statistical Classification of Diseases and Related Health Problems (ICD) and Current Procedural Technology (CPT) code prioritization system code prioritization system of claim 13, wherein RVUs are generated and stored locally in a database in a memory or are accessed from an external system via the Internet.

15. The International Statistical Classification of Diseases and Related Health Problems (ICD) and Current Procedural Technology (CPT) code prioritization system code prioritization system of claim 14, wherein actual measured CPT codes and IDS codes ranking algorithms increment, decrement, order and rank based on increased probability of third-party acceptance, those claims which are more closely tailored to represent the care a patient receives, and/or those codes which are provider-specific, practice-specific or location-specific.

16. The International Statistical Classification of Diseases and Related Health Problems (ICD) and Current Procedural Technology (CPT) code prioritization system of claim 15, wherein ICD and CPT codes are provided via a user interface as prioritized, preferenced or incremented codes for provider selection based on provider use, provider type, care location, location type, incremented codes, reimbursement rates, or a combination thereof, based on one or a number of factors including provider utilization, specialty field, a specific provider, a provider type, congruence with a billing module, billing specialists self-population, auto-population, provider location, algorithm selection based on accepted or rejected claims, for expediance and ease of access.

17. The International Statistical Classification of Diseases and Related Health Problems (ICD) and Current Procedural Technology (CPT) code prioritization system of claim 16, wherein the server calculates the differences, standard deviation, average, or interpolation for prioritized, preferenced or incremented ICD codes CPT codes, CPT RVU values, or a combination thereof, for provider selection or selections to set or modify thresholds, via criteria selection or a feedback loop, for ranking and ordering.

18. The International Statistical Classification of Diseases and Related Health Problems (ICD) and Current Procedural Technology (CPT) code prioritization system of claim 11 wherein CPT code prioritization control logic can generate, via the server, a list or subset of lists for prioritized, preferenced or incremented codes for provider selection or selections using the selected ICD codes CPT codes, or a combination of both.

19. The International Statistical Classification of Diseases and Related Health Problems (ICD) and Current Procedural Technology (CPT) code prioritization system of claim 18 wherein ICD codes, baseline ICD codes, CPT codes and CPT RVU values for prioritized, preferenced or incremented codes are preferentially displayed for selection from all or a subset of available codes based on provider use, provider type, care location, location type, incremented codes, reimbursement rates, or a combination thereof, based on one or a number of factors including utilization, specialty field, a specific provider, a provider type, congruence with a billing module, provider or billing specialists self-population, auto-population, provider location, algorithm selection based on accepted or rejected claims, reimbursement rates, or both, for expedience and ease of access.

20. The International Statistical Classification of Diseases and Related Health Problems (ICD) code prioritization system of claim 18 wherein provider encounter time is captured, collected and monitored as to ensure proper billing between and among providers as a function of a CPT code selection, CPT RVU selectable rate or rates, or a combination thereof, for proper billing and reimbursement optimization.
